(19) United States
(12) Patent Application Publication
(10) Pub. No.: US 2004/0038854 A1
(43) Pub. Date: Feb. 26, 2004

(54) Use of il-8 receptor antagonists in the treatment of virus infections

(76) Inventors: Susan B. Dillon, Alamo, CA (US); Ruth Tal-Singer, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/333,095

(22) PCT Filed: Jul. 16, 2001
(86) PCT No.: PCT/US01/22328

(51) Int. Cl. A61K 31/00

(52) U.S. Cl. 514/1

(57) ABSTRACT

The present invention is directed to the novel use of an IL-8 receptor antagonist for the treatment of human virus infections and the exacerbation of symptoms associated therewith.

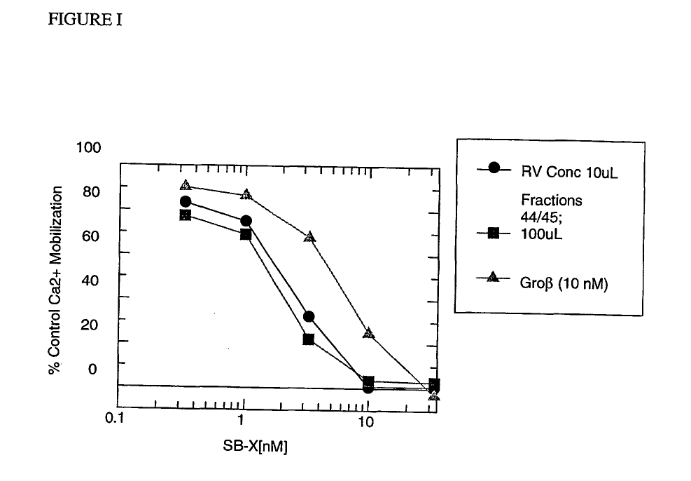

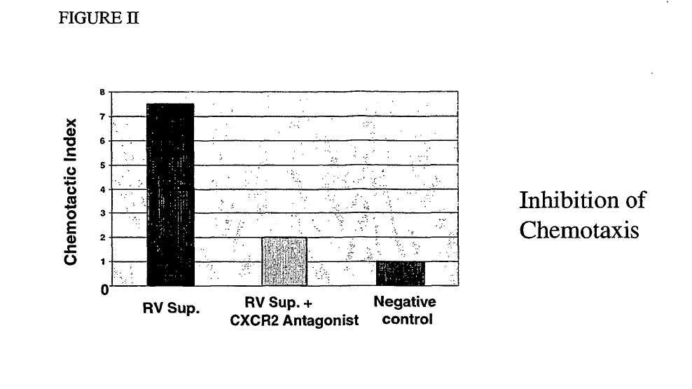

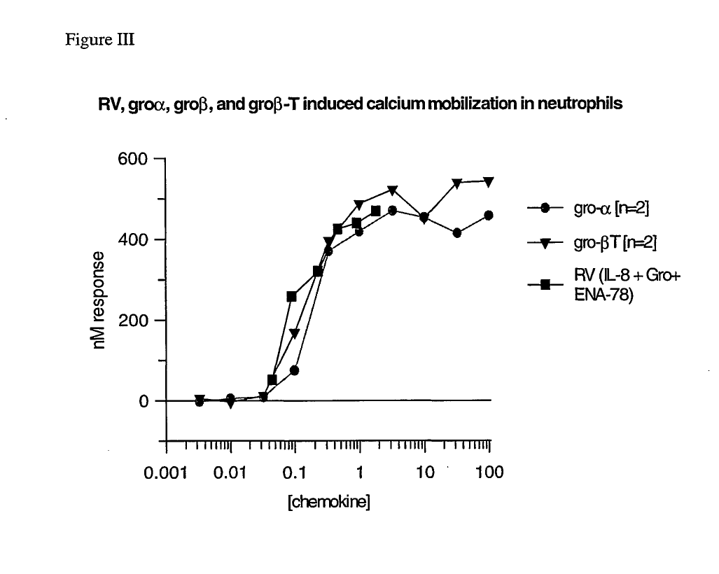

### FIELD OF THE INVENTION

This invention relates to the use of IL-8, GROα, GROβ, GROγ, NAP-2, and ENA-78 modulators in the treatment of viral infections.

### BACKGROUND OF THE INVENTION

Human rhinovirus (HRV), the most frequent cause of the common cold, is increasingly associated with more serious sequelae including exacerbations of asthma, chronic bronchitis, COPD, otitis media, and sinusitis. Recent published studies in adults and adolescents, using PCR to assist in viral detection, have shown that up to 50 to 80% of asthma exacerbation's are associated with upper respiratory tract virus infection, and that rhinovirus is the most common cold virus.

HRV infects nasal epithelial cells. Recent evidence suggests the virus may also infect bronchial epithelium. Prodromal cold symptoms are apparent within 24 hours post-infection, peak on days 2 through 5, and resolve within 7 to 14 days. However, the effects can be more protracted in some individuals. While the virus may clear, symptoms often persist. Symptoms are believed to arise more from the host's response to infection, than an acute cytotoxic effect, since only a small fraction of upper respiratory epithelial cells are demonstrably infected, and there is minimal epithelial cell damage. Increased intranasal levels of kinins, IL-1, IL-8, IL-6, IL-11, and neutrophils are found in normal individuals infected with rhinoviruses. A correlation between IL-8 concentration in nasal secretions with local myeloperoxidase level as well as symptom severity has been demonstrated in several recent studies. Intranasal concentrations of IL-1 and IL-6 have been correlated with symptom severity as well. Experimental rhinovirus infection also results in enhanced immediate and late phase allergic reactions, and in increased infiltration of T lymphocytes and eosinophils into the lower airways. In atopics and asthmatics, these effects persist for up to 2 months post-infection. Human bronchial epithelial cell lines have been shown to produce IL-1, IL-6, IL-8, IL-11 and GM-CSF in response to rhinovirus infection. Early production of cytokines by rhinovirus-infected epithelial cells may therefore be responsible for triggering recruitment of neutrophils, T-cells and activated eosinophils into the upper and lower airways.

Additionally, IL-1, IL-6 and IL-8 are also produced in response to infection with other respiratory viruses (influenza, respiratory syncytial virus) which can cause the common cold and associated sequelae.

Many different names have been applied to Interleukin-8 (IL-8), such as neutrophil attractant/activation protein-1 (NAP-1), monocyte derived neutrophil chemotactic factor (MDNCF), neutrophil activating factor (NAF), and T-cell lymphocyte chemotactic factor. Interleukin-8 is a chemoattractant for neutrophils, basophils, and a subset of T-cells. It is produced by a majority of nucleated cells including macrophages, fibroblasts, endothelial and epithelial cells exposed to TNF, IL-1α, IL-1β or LPS, and by neutrophils themselves when exposed to LPS or chemotactic factors such as FMLP. M. Baggiolini et al., J. Clin. Invest. 84, 1045 (1989); J. Schroder et al, J. Immunol. 139, 3474 (1987) and J. Immunol. 144, 2223 (1990); Strieter, et al., Science 243, 1467 (1989) and J. Biol. Chem. 264, 10621 (1989); Cassatella et al., J. Immunol. 148, 3216 (1992).

GROα, GROβ, GROγ and NAP-2 also belong to the chemokine family. Like IL-8 these chemokines have also been referred to by different names. For instance GROα, β, γ have been referred to as MGSAα, β and γ respectively (Melanoma Growth Stimulating Activity), see Richmond et al., J. Cell Physiology 129, 375 (1986) and Chang et al., J. Immunol 148, 451 (1992). All of the chemokines of the α-family which possess the ELR motif directly preceding the CXC motif bind to the EL-8 B receptor (CXCR2).

IL-8, GROα, GROβ, GROγ, NAP-2, and ENA-78 stimulate a number of functions in vitro. They have all been shown to have chemoattractant properties for neutrophils, while IL-8 and GROα have demonstrated T-lymphocytes, and basophilic chemotactic activity. In addition IL-8 can induce histamine release from basophils from both normal and atopic individuals. GRO-α and IL-8 can in addition, induce lysozomal enzyme release and respiratory burst from neutrophils. IL-8 has also been shown to increase the surface expression of Mac-1 (CD11b/CD18) on neutrophils without de novo protein synthesis.

In vitro, IL-8, GROα, GROβ, GROγ and NAP-2 induce neutrophil shape change, chemotaxis, granule release, and respiratory burst, by binding to and activating receptors of the seven-transmembrane, G-protein-linked family, in particular by binding to IL-8 receptors, most notably the IL-8β receptor (CXCR2). Thomas et al., J. Biol. Chem. 266, 14839 (1991); and Holmes et al., Science 253, 1278 (1991).

Two high affinity human IL-8 receptors (77% homology) have been characterized: IL-8RA, which binds only IL-8 with high affinity, and IL-8RB, which has high affinity for IL-8 as well as for GROα, GROβ, GROγ and NAP-2. See Holmes et al., supra; Murphy et al., Science 253, 1280 (1991); Lee et al., J. Biol. Chem. 267, 16283 (1992); LaRosa et al., J. Biol. Chem. 267, 25402 (1992); and Gayle et al., J. Biol. Chem. 268, 7283 (1993).

Interference with the biochemical processes of epithelial cells resulting from virus infection represents a viable new therapeutic target for an IL-8 receptor antagonist. The present invention is directed to the novel discovery of treatment of this therapeutic target.

### SUMMARY OF THE INVENTION

The present invention provides for a method of treating a chemokine mediated disease, wherein the chemokine is one which binds to an IL-8 A or B receptor and which method comprises administering an effective amount of an IL-8 receptor antagonist, or a pharmaceutically acceptable salt thereof. The present invention further relates to the use of an IL-8 receptor antagonist for the treatment, including prophylaxis and prevention/reduction of the severity of the underlying condition, of a virus infection including but not limited to human rhinovirus, enteroviruses, coronavirus, influenza virus, parainfluenza virus, respiratory syncytial virus, and adenovirus, in a human in need thereof, which method comprises administering to said human an effective amount of an IL-8 receptor antagonist.

### DETAILED DESCRIPTION OF THE INVENTION

IL-8 and other cytokines affect a wide variety of cells and tissues and these cytokines as well as other leukocyte derived cytokines are important and critical inflammatory mediators responsible for the symptoms of many viral infections. The inhibition of these cytokines is of benefit in controlling, reducing and alleviating many of these symptoms of respiratory viral infection. In addition, the present invention is directed to the treatment of symptoms caused by viral infection in a human which is caused by the human rhinovirus, other enterovirus, coronavirus, herpesviruses, influenza virus, parainfluenza virus, respiratory syncytial virus or an adenovirus. In addition, the present invention is directed to respiratory viral infections, which exacerbate underlying chronic conditions such as asthma, chronic bronchitis, chronic obstructive pulmonary disease, otitis media, and sinusitis. It should also be noted that the respiratory viral infection treated herein may be associated with a secondary bacterial infection, such as otitis media, sinusitis or pneumonia.

The present invention will demonstrate that IL-8 receptor antagonists are useful in the treatment of symptoms associated with respiratory viral infection and prevention/reduction of the severity of exacerbations of underlying conditions, including asthma and otitis media, COPD, sinusitis, chronic bronchitis, etc amongst others.

Suitable IL-8 inhibitors are well known in the art, and an assay for determining IL-8 inhibition is also readily available. For instance, see U.S. Pat. Nos. 5,886,044, 5,780,483, 6,005,008, 5,929,250, 6,015,908; 5,919,776, U.S. application Ser. Nos. 09/111663, 09/125279, 09/240354, 09/202570, 09/202586, 09/202569, 09/202568, 09/230120, 09/230290, 09/230952, 09/230977, 09/230981, 09/230980, 09/242187, 09/341378, 09/341382, 09/341262, 09/463673, 09/508039, 09/486986, WO99/65310, WO 0012489, WO 0009511, WO 9942464, WO 9942463, WO 9942461, WO00/05216, WO99/36069, WO99/36070, WO00/06557, PCT/US99/23776,PCT/US99/29940, U.S. Provisional Applications 60/134728,60/136666,60/136665,60/136717,60/136667,60/139675, 60/139680,60/139678,60/139673,60/140024,60/139677,60/139674, 60/140025,60/145756,60/164350,60/186239,60/186183,60/186182, 60/188410,60/188243,60/189176,60/189175,60/189848,60/192132, or 60/196022.

The IL-8 receptor antagonist may also be administered with a second therapeutic agent. The second therapeutic agent may be an antiviral agent such as ribavirin, amantidine, rimantidine, relenza, tamiflu, BTA 188, RWJ-270210 (BCX-1812), sICAM-1, tICAM453, Pleconaril or AG 7088; it may also be an antihistamine, such as Benadryl, chlorpheneramine and salts thereof, brompheneramine or salts thereof, etc, a decongestant, such as phenylpropanolamine and salts thereof, pseudoephedreine or salts thereof; steroids, such as dexamethasone, prednisone, or prednisolone, etc; various antibiotics, such as the quinolones, cephalosporins, β-lactamase inhibitors, etc.; anti-inflammatory agents, such as CSAIDS, COX-1 or COX-2 inhibitors, ASA, or indomethacin, etc.

### DESCRIPTION OF THE FIGURES

FIG. I—Inhibition of RV or Gro-beta induced calcium mobilization in human neutrophils by CXCR2 antagonist X.

FIG. II—Inhibition of RV-induced chemotaxis of human neutrophils by CXCR2 antagonist X.

FIG. III—RV, groα, groβ, and groβ-T induced calcium mobilization in neutrophils.

### METHOD OF TREATMENT

The present IL-8 receptor antagonist, or a pharmaceutically acceptable salt thereof, can be used in the manufacture of a medicine for the prophylactic or therapeutic treatment of symtoms or sequelae of viral infection in a human, or other mammal, which is exacerbated or caused by excessive or unregulated IL-8 cytokine production by such mammal's cell, such as but not limited to monocytes and/or macrophages, or other chemokines which bind to the IL-8 A or B receptor, also referred to as the type I or type II receptor.

Accordingly, the present invention provides a method of treating a viral infection, wherein the chemokine is one which binds to an IL-8 A or B receptor and which method comprises administering an effective amount of a present inhibitor or a pharmaceutically acceptable salt thereof. In particular, the chemokines are IL-8, GROα, GROβ, GROγ, NAP-2 or ENA-78.

In order to use a present IL-8 receptor antagonist or a pharmaceutically acceptable salt thereof in therapy, it will normally be formulated into a pharmaceutical composition in accordance with standard pharmaceutical practice. This invention, therefore, also relates to a pharmaceutical composition comprising an effective, non-toxic amount of a present IL-8 receptor antagonist and a pharmaceutically acceptable carrier or diluent.

The present IL-8 receptor antagonists, pharmaceutically acceptable salts thereof and pharmaceutical compositions incorporating such may conveniently be administered by any of the routes conventionally used for drug administration, for instance, orally, topically, bucolally, parenterally or by inhalation. They may be administered in conventional dosage forms prepared by combining a present compound with standard pharmaceutical carriers according to conventional procedures. They may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical carrier employed may be, for example, either a solid or liquid. Exemplary of solid carriers are lactose, terra alba, sucrose, talc, gelatin, agar, pectin, acacia,.magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquid such as an ampule or nonaqueous liquid suspension.

The present antagonists may be administered topically, that is by nonsystemic administration. This includes the application of a present compound externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as liniments, lotions, creams, ointments or pastes, and drops suitable for administration to the ear or nose, solutions or suspensions suitable for inhalation. The active ingredient may comprise, for topical administration, from 0.001% to 10% w/w, for instance from 1% to 2% by weight of the Formulation. It may however comprise as much as 10% w/w but preferably will comprise less than 5% w/w, more preferably from 0.1% to 1% w/w of the Formulation.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

The present antagonists may be administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The present antagonists may also be administered by inhalation that is by intranasal and oral inhalation administration. Appropriate dosage forms for such administration, such as an aerosol formulation or a metered dose inhaler, may be prepared by conventional techniques.

For all methods of use disclosed herein for the present antagonists the daily oral dosage regimen will preferably be from about 0.01 to about 80 mg/kg of total body weight. The daily parenteral dosage regimen about 0.001 to about 80 mg/kg of total body weight. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of a present antagonist or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a present compound or a pharmaceutically acceptable salt thereof given per day for a defined number of days, can be ascertained by those skilled in the art using conventional course of treatment determination tests.

The invention will now be described by reference to the following biological examples, which are merely illustrative and are not to be construed as a limitation of the scope of the present invention.

### BIOLOGICAL EXAMPLES

The IL-8, and GRO-α chemokine inhibitory effects of compounds of the present invention are determined by the following in vitro assay:

Receptor Binding Assays:

[¹²⁵I] IL-8 (human recombinant) is obtained from Amersham Corp., Arlington Heights, Ill., with specific activity 2000 Ci/mmol. GRO-α is obtained from NEN-New England Nuclear. All other chemicals are of analytical grade. High levels of recombinant human IL-8 type α and β receptors were individually expressed in Chinese hamster ovary cells as described previously (Holmes, et al., Science, 1991, 253, 1278). The Chinese hamster ovary membranes were homogenized according to a previously described protocol (Haour, et al., J. Biol. Chem., 249 pp 2195-2205 (1974)). Except that the homogenization buffer is changed to 10 mM Tris-HCL, 1 mM MgSO₄, 0.5 mM EDTA (ethylene-diaminetetraacetic acid), 1 mM PMSF (α-toluenesulphonyl fluoride), 0.5 mg/L Leupeptin, pH 7.5. Membrane protein concentration is determined using Pierce Co. micro-assay kit using bovine serum albumin as a standard. All assays are performed in a 96-well micro plate format. Each reaction mixture contains ¹²⁵I IL-8 (0.25 nM) or ¹²⁵I GRO-α and 0.5 μg/mL of IL-8Rα or 1.0 μg/mL of IL-8Rβ membranes in 20 mM Bis-Trispropane and 0.4 mM Tris HCl buffers, pH 8.0, containing 1.2 mM MgSO₄, 0.1 mM EDTA, 25 mM Na and 0.03% CHAPS. In addition, drug or compound of interest is added which has been pre-dissolved in DMSO so as to reach a final concentration of between 0.01 nM and 100 uM. The assay is initiated by addition of ¹²⁵I-IL-8. After 1 hour at room temperature the plate is harvested using a Tomtec 96-well harvester onto a glass fiber filtermat blocked with 1% polyethylenimine/0.5% BSA and washed 3 times with 25 mM NaCl, 10 mM TrisHCl, 1 mM MgSO₄, 0.5 mM EDTA, 0.03% CHAPS, pH 7.4. The filter is then dried and counted on the Betaplate liquid scintillation counter. The recombinant EL-8 Rα, or Type I, receptor is also referred to herein as the non-permissive receptor and the recombinant IL-8 Rβ, or Type II, receptor is referred to as the permissive receptor.

Chemotaxis Assay:

The in vitro inhibitory properties of these compounds are determined in the neutrophil chemotaxis assay as described in Current Protocols in Immunology, vol. I, Suppl 1, Unit 6.12.3., whose disclosure is incorporated herein by reference in its entirety. Neutrophils where isolated from human blood as described in Current Protocols in Immunology Vol. I, Suppl 1 Unit 7.23.1, whose disclosure is incorporated herein by reference in its entirety. The chemoattractants IL-8, GRO-α, GRO-β, GRO-γ and NAP-2 are placed in the bottom chamber of a 48 multiwell chamber (Neuro Probe, Cabin John, Md.) at a concentration between 0.1 and 100 nM. The two chambers are separated by a 5 uM polycarbonate filter. When compounds of this invention are tested, they are mixed with the cells (0.001-1000 nM) just prior to the addition of the cells to the upper chamber. Incubation is allowed to proceed for between about 45 and 90 min at about 37° C. in a humidified incubator with 5% CO₂. At the end of the incubation period, the polycarbonate membrane is removed and the top side washed, the membrane then stained using the Diff Quick staining protocol (Baxter Products, McGaw Park, Ill., USA). Cells which have chemotaxed to the chemokine are visually counted using a microscope. Generally, four fields are counted for each sample, these numbers are averaged to give the average number of cells which had migrated. Each sample is tested in triplicate and each compound repeated at least four times. To certain cells (positive control cells) no compound is added, these cells represent the maximum chemotactic response of the cells. In the case where a negative control (unstimulated) is desired, no chemokine is added to the bottom chamber. The difference between the positive control and the negative control represents the chemotactic activity of the cells.

Elastase Release Assay:

The compounds of this invention are tested for their ability to prevent Elastase release from human neutrophils. Neutrophils are isolated from human blood as described in Current Protocols in Immunology Vol. I, Suppl 1 Unit 7.23.1. PMNs 0.88×10⁶ cells suspended in Ringer's Solution (NaCl 118, KCl 4.56, NaHCO₃ 25, KH₂PO₄ 1.03, Glucose 11.1, HEPES 5 mM, pH 7.4) are placed in each well of a 96 well plate in a volume of 50 ul. To this plate is added the test compound (0.001-1000 nM) in a volume of 50 ul, Cytochalasin B in a volume of 50 ul (20 ug/ml) and Ringers buffer in a volume of 50 ul. These cells are allowed to warm (37° C., 5% CO2, 95% RH) for 5 min before IL-8, GROα, GROβ, GROγ or NAP-2 at a final concentration of 0.01-1000 nM was added. The reaction is allowed to proceed for 45 min before the 96 well plate is centrifuged (800×g 5 min.) and 100 ul of the supernatant removed. This supernatant is added to a second 96 well plate followed by an artificial elastase substrate (MeOSuc-Ala-Ala-Pro-Val-AMC, Nova Biochem, La Jolla, Calif.) to a final concentration of 6 ug/ml dissolved in phosphate buffered saline. Immediately, the plate is placed in a fluorescent 96 well plate reader (Cytofluor 2350, Millipore, Bedford, Mass.) and data collected at 3 min intervals according to the method of Nakajima et al J. Biol. Chem. 254 4027 (1979). The amount of Elastase released from the PMNs is calculated by measuring the rate of MeOSuc-Ala-Ala-Pro-Val-AMC degradation.

Rhinovirus Methods:

Cell lines and rhinovirus serotype 39 were purchased from American Type Culture Collection (ATCC). BEAS-2B cells were cultured according to instructions provided by ATCC using BEGM (bronchial epithelial growth media) purchased from Clonetics Corp. HELA cell cultures, used for detection and titration of virus, were maintained in Eagle's minimum essential media containing 10% fetal calf serum, 2 mM 1-glutamine, and 10 mM HEPES buffer (MEM).

A modification of the method reported by Subauste et al., Supra, for in vitro infection of human bronchial epithelial cells with rhinovirus was used in these studies. BEAS-2B cells (2×10⁵/well) were cultured in collagen-coated wells for 24 hours prior to infection with rhinovirus. Rhinovirus serotype 39 was added to cell cultures for one hour incubation at 34° C. after which inoculum was replaced with fresh media and cultures were incubated for an additional 72 hours at 34° C. Supernatants collected at 72 hours post-infection were assayed for cytokine protein concentration by ELISA using commercially available kits (R&D Systems). Virus yield was also determined from culture supernatants using a microtitration assay in HELA cell cultures (Subauste et aL, supra 1995). In cultures treated with IL-8 inhibitors, drug was added 30 minutes prior to infection. Stocks of compounds were prepared in DMSO (10 mM drug) and stored at −20° C.

For detection of IL-8R inhibition, cultures were incubated in basal media without growth factors and additives to reduce endogenous levels of activated IL-8. Supernatants were harvested at various time points after addition of rhinovirus and concentrated. Concentrates were fractionated on Superose 6 columns. Supernatants were concetrated >50 fold using an Amicon concetrater with 5,000 mol. wt. cut off. A singel injection (0.5 ml) was applied to a Superose 6 size fractionation column which was eluted at a singel flow rate (0.2 ml/min). 0.5 ml fractions were collaceted and assayed for Ca2⁺ mobilizing activity in freshly isolated human PMN loaded with FURA-2.

Results:

Characterization of Chemokines Produced from RV Infected BEAS-2B Human Epithelial Cells.

Under resting conditions human BEAS-2B human epithelial cells produced small quantities of at least three known human chemokines, Groα, IL-8 and ENA-78. When these cells are infected with rhinovirus production of the three ELR chemokines increases 6.6-20 fold above resting conditions (Table 1). Table 1. Production of ELR chemokines from BEAS-2B epithelial cells under resting and infected conditions (n=6).

| Treatment | Groa (pg/ml) | IL-8 (pg/ml) | ENA-78 (pg/ml) |
|---|---|---|---|
| Control | 1568 ± 402 | 143 ± 86 | 164 ± 33 |
| HRV-39 | 12135 ± 3599 | 2870 ± 645 | 1083 ± 194 |

When HRV-39 infected epithelial cell supernatant is subject to size exclusion fractionation over a superose 6 column it results in a single peak of Ca2⁺ mobilizing activity when assayed using human neutrophils (FIG. III) or a cell line transfected with either the CXCR1 or CXCR2 receptor. The mobilization of Ca2⁺ in the neutrophil occurs in the same fractions as the elution of the chemokines IL-8, ENA-78 and Groα.

To determine if other chemokines are produced from HRV-39 infected BEAS-2B for which we do not have ELISA kits, BEAS-2B supernatant was assayed against other freshly isolated human peripheral cells which express a variety of chemokine receptors including CCR1, CCR2, CCR3 and CCR5. When the above active fractions derived from the BEAS-2B epithelial cells were assayed for Ca2⁺ mobilization using Fura-2 loaded eosinophils or peripheral blood mononuclear cells (PBLs) none of the fractions mobilized Ca2⁺. This indicates that chemokines besides IL-8, Groα and ENA-78 are not present at significant concentrations to promote Ca2⁺ mobilization.

To determine the potential for other PMN activating chemokines and for the possibility that IL-8 will activate the PMNs via CXCR2 we have determined the ability of IL-8 receptor antagonists to inhibit Ca2⁺ mobilization induced by groβ, concentrated BEAS-2B supernatant or active fractions derived from a superose 6 column separation in Fura-2 loaded PMNs. As can be seen from FIG. I, compound-X dose dependently and completely inhibited RV concentrated supernatant and fractions containing the chemokines from a superose 6 column with IC₅₀s between 1.7 and 2 nM. This IC₅₀ is similar to the IC₅₀ derived with Groβ alone, (IC50=5 nM) and indicates that all of the Ca2⁺ mobilizing activity contained in the concentrate or fractions is working through the CXCR2 receptor.

To determine if the RV supernatant works solely through the CXCR2 receptor, we determined that rank correlation of a number of selective CXCR2 antagonist for there ability to inhibit Ca2⁺ mobilization induced by concentrated RV supernatant and Groβ. There is an excellent rank correlation for a divergent set of CXCR2 antagonist and their ability to inhibit Ca2⁺ mobilization induced by RV and Grob, indicating that RV supernatant Ca2⁺ mobilizing activity works solely through the CXCR2 receptor on PMNs.

Inhibition of PMN chemotaxis was also determined in both the concentrated and fractionated RV sample. Chemotaxis was inhibited by specific CXCR2 antagonists as shown in FIG. II.

These results demonstrate that BEAS-2B cells produce a variety of ELR chemokines which act through the CXCR2 receptor and which can be blocked by selective CXCR2 antagonist. Although IL-8 is present in the supernatant it also is blocked completely by the CXCR2 antagonist. This is improbably do to its low level compared with Groα.

All publications, including but not limited to patents and patent applications, cited in this specification are herein incorporated by reference as if each individual publication were specifically and individually indicated to be incorporated by reference herein as though fully set forth.

The above description filly discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A method of treating symptomology of the common cold as caused by human rhinovirus, other enterovirus, herpesvirus, coronavirus, influenza virus, parainfluenza virus, respiratory syncytial virus, or adenovirus infection in a human in need thereof which method comprises administering to said human an effective amount of an IL-8 receptor antagonist.

2. The method according to claim 1 wherein the respiratory viral infection exacerbates asthma.

3. The method according to claim 1 wherein the respiratory viral infection exacerbates chronic bronchitis.

4. The method according to claim 1 wherein the respiratory viral infection exacerbates chronic obstructive pulmonary disease.

5. The method according to claim 1 wherein the respiratory viral infection exacerbates otitis media.

6. The method according to claim 1 wherein the respiratory viral infection exacerbates sinusitis.

7. The method according to claim 1 wherein the respiratory viral infection is associated with a second bacterial infection, such as otitis media, sinusitis or pneumonia.

8. The method according to any one of claims 1 to 7 wherein the IL-8 receptor antagonist is administered with a second therapeutic agent.

9. The method according to claim 1 wherein the second therapeutic agent is selected from the group consisting of an antiviral agent, an antihistamine, a decongestant, a steroid, an antibiotic, and an anti-inflammatory agent.

10. The method according to any one of claims 1-9 wherein the therapeutic agent is administered orally, bucolally, topically (intranasally) or via inhalation (aerosol), or both topically and via inhalation.

11. The method according to claim 10 wherein the compound is administered with a second therapeutic agent.

12. The method according to claim 11 wherein the second therapeutic agent is selected from the group consisting of an antiviral agent; an antihistamine; a decongestant; a steroid; an antibiotic, and an anti-inflammatory agent.

13. The method according to claim 1 wherein the IL-8 receptor antagonist is selected from a compound disclosed in:
U.S. Pat. Nos. 5,886,044, 5,780,483, 6,005,008, 5,929,250, 6,015,908; or 5,919,776, U.S. application Ser. Nos. 09/111663, 09/125279, 09/240354, 09/202570, 09/202586, 09/202569, 09/202568, 09/230120, 09/230290, 09/230952, 09/230977, 09/230981, 09/230980, 09/242187, 09/341378, 09/341382, 09/341262, 09/463673, 09/508039, or 09/486986, WO99/65310, WO 0012489, WO 0009511, WO 9942464, WO 9942463, WO 9942461, WO00/05216, WO99/36069, WO99/36070, or WO00/06557, PCT/US99/23776, or PCT/US99/29940, U.S. Provisional Application 60/134728,60/136666,60/136665,60/136717,60/136667,60/139675, 60/139680,60/139678,60/139673,60/140024,60/139677,60/139674, 60/140025,60/145756,60/164350,60/186239,60/186183,60/186182, 60/188410,60/188243,60/189176,60/189175,60/189848,60/192132, or 60/196022.
